(19) United States
(12) Patent Application Publication
(10) Pub. No.: US 2015/0297343 A1
(43) Pub. Date: Oct. 22, 2015

(54) IMPROVED INTRAOCULAR LENS AND CORRESPONDING MANUFACTURING METHOD

(71) Applicant: Frederic HEHN, Nancy (FR)

(72) Inventor: Frederic HEHN, Nancy (FR)

(21) Appl. No.: 14/373,980

(22) PCT Filed: Jan. 22, 2013
(86) PCT No.: PCT/FR2013/050133

§ 371 Date: Jul. 23, 2014

(30) Foreign Application Priority Data
Jan. 24, 2012 (FR) 1200190

(51) Int. Cl. A61F 2/16; A61B 3/00

(52) U.S. Cl. CPC A61F 2/1618; A61B 3/0025

(57) ABSTRACT

An intraocular lens has an optical axis, a central zone and a peripheral zone which are substantially symmetrical with respect to the optical axis and which extend substantially perpendicular thereto, the central zone extending to a first distance, and the peripheral zone extending from the first distance to the end of the intraocular lens, wherein the central zone has a nominal optical power, and the peripheral zone has a radius of curvature which varies continuously and monotonously as a function of the distance to the optical axis, so that a target asphericity value is obtained at a second distance relative to the optical axis, the first distance and the second distance being calculated from a photopic pupil diameter and a mesopic pupil diameter, respectively, of a patient.

The invention relates to the field of ophthalmology, and more particularly to intraocular lenses.

The field of intraocular lenses has known many discoveries and much progress over the past ten years. Indeed, the treatment of cataracts has become a conventional operation which has been mastered.

However, this field remains a field that is at the forefront of research, and in which the maturity of the methods remains relative. This is reflected especially in the fact that there is as yet no intraocular lens which allows both myopia (or hypermetropia) and presbyopia to be corrected satisfactorily. Indeed, the only implants which aim to solve this problem are multifocal lenses, which are the source of halos which can be very annoying.

The invention will improve the situation.

To that end, the invention proposes an intraocular lens, characterised in that it has an optical axis and a central zone and a peripheral zone which are substantially symmetrical with respect to said optical axis and which extend substantially perpendicular thereto, said central zone extending to a first distance, and the peripheral zone extending from the first distance to the end of the intraocular lens, wherein the central zone has a nominal optical power, and the peripheral zone has a radius of curvature which varies continuously and monotonously as a function of the distance to the optical axis, so that a target asphericity value is obtained at a second distance relative to the optical axis, the first distance and the second distance being calculated from a photopic pupil diameter and a mesopic pupil diameter, respectively, of a patient.

The invention relates also to a method for calculating a radius of curvature profile for an intraocular lens, which method comprises the following steps:
a) receiving biometric parameters of a patient, comprising at least a first radius of curvature, a photopic pupil diameter and a mesopic pupil diameter,
b) determining an emmetropic distance from at least the mesopic pupil diameter, and a second radius of curvature from the first radius of curvature and a target asphericity value,
c) calculating for the intraocular lens a desired radius of curvature profile in a direction substantially perpendicular to an optical axis, wherein the radius of curvature is equal to the first radius of curvature in a central zone extending between the optical axis and a first distance calculated from at least the photopic pupil diameter, and wherein, in a peripheral zone extending from the first distance to the end of the intraocular lens, the radius of curvature varies continuously and monotonously as a function of the distance to the optical axis, so that the radius of curvature is equal to the second radius of curvature at the emmetropic distance relative to the optical axis.

Other features and advantages of the invention will better become apparent upon reading the following description, which is taken from examples which are given by way of illustration and without implying any limitation and are taken from the drawings, in which:

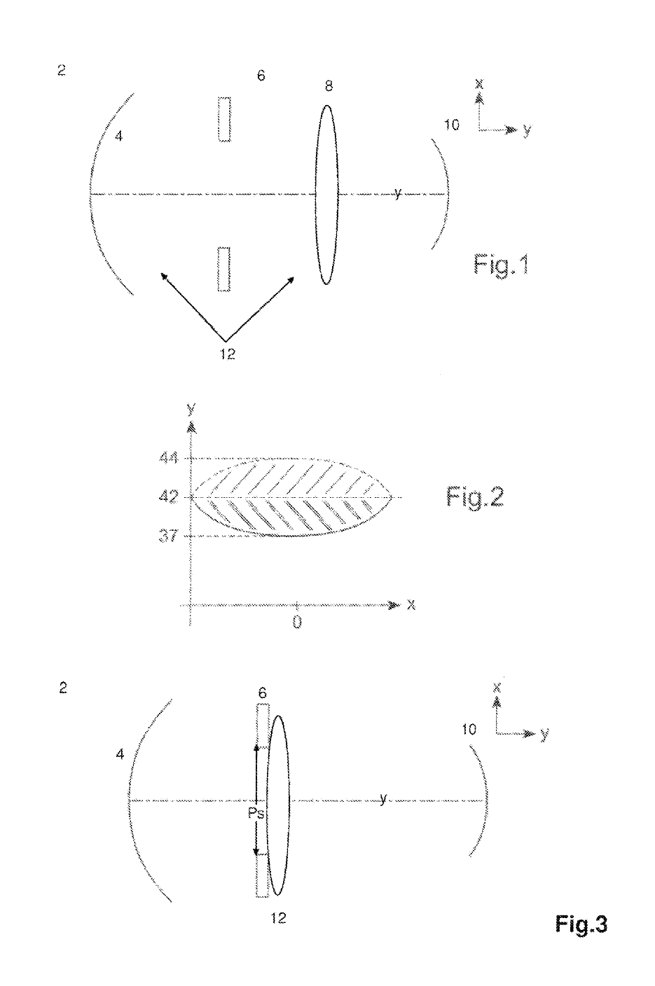

FIG. 1 shows an optical diagram of an eye,

FIG. 2 shows three keratometric profiles of an eye,

FIG. 3 shows a schematic view of an eye in which an intraocular lens according to the invention is implanted, and in which the pupil is dilated to the maximum,

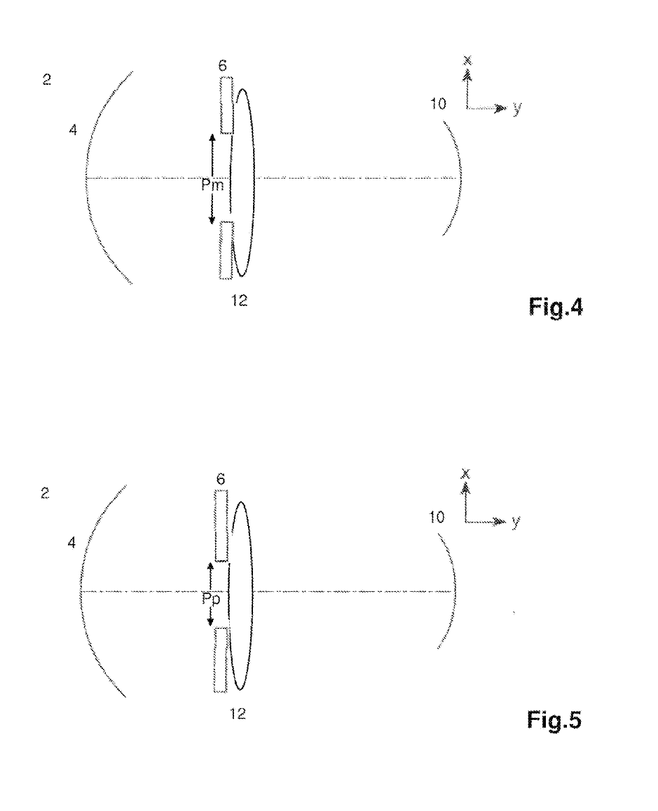

FIG. 4 shows a schematic view of an eye in which an intraocular lens according to the invention is implanted, and in which the pupil is moderately dilated,

FIG. 5 shows a schematic view of an eye in which an intraocular lens according to the invention is implanted, and in which the pupil is dilated to the minimum,

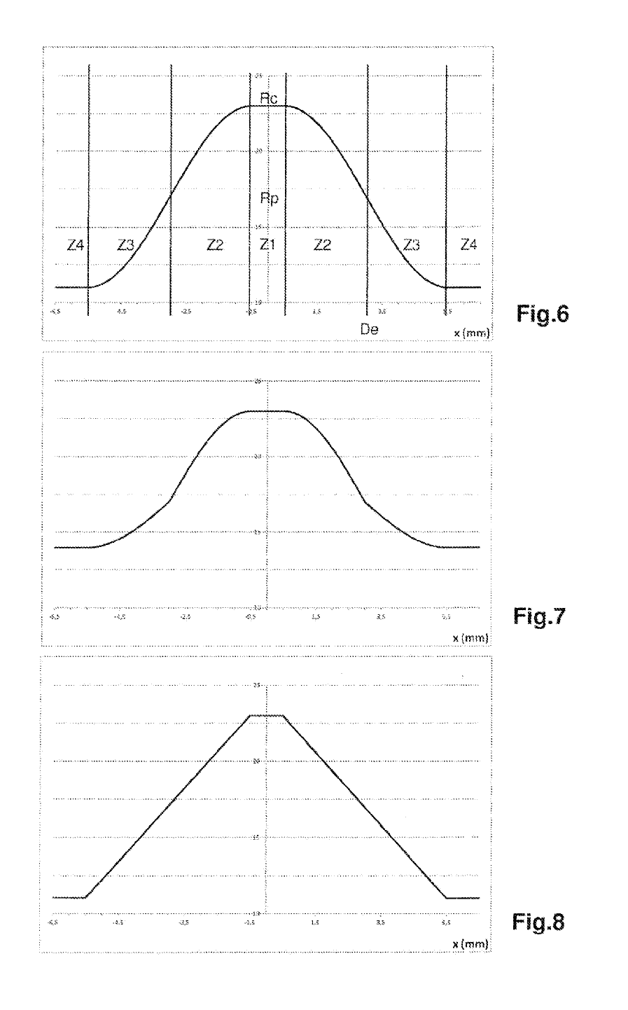

FIG. 6 shows a diagram of the radius of curvature profile of the lens of FIGS. 3 to 5,

FIG. 7 shows a diagram of the radius of curvature profile of an alternative embodiment of an intraocular lens according to the invention,

FIG. 8 shows a diagram of the radius of curvature of an alternative embodiment of an intraocular lens according to the invention,

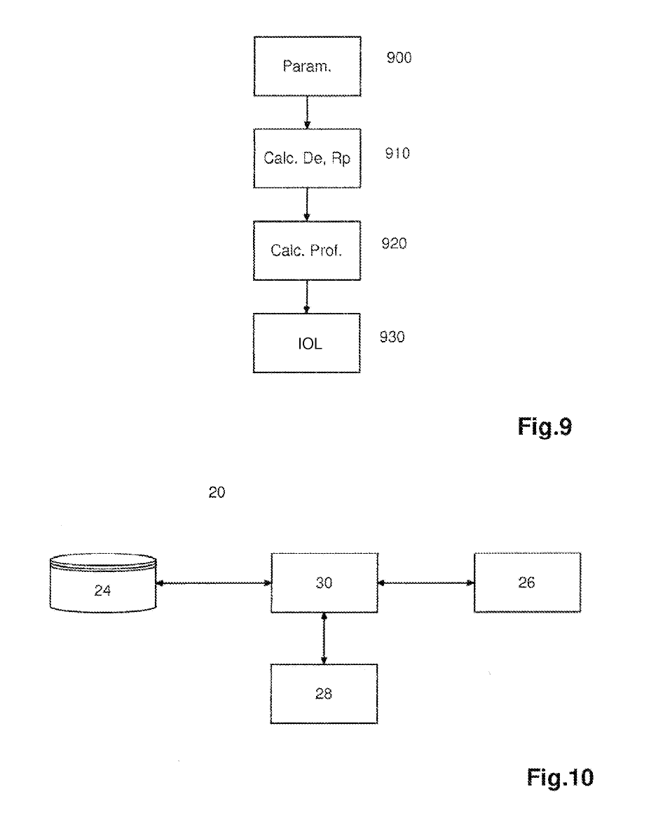

FIG. 9 shows a flow diagram as an example of a method for manufacturing an intraocular lens according to the invention, and

FIG. 10 shows a diagram of a device for calculating an intraocular lens profile according to the invention, which can be employed in the method of FIG. 9.

The drawings and the description below mainly contain elements of a specific nature. They may therefore not only serve for better understanding of the present invention but also contribute to the definition thereof, where appropriate.

The detailed description is further supplemented by annex A, which gives the formulation of some mathematical formulae employed within the scope of the invention. This Annex is set apart with the aim of clarification, and to facilitate cross-referencing. It is an integral part of the description and may therefore not only serve for better understanding of the present invention but also contribute to the definition thereof, where appropriate.

FIG. 1 shows an optical diagram allowing the vision in an eye to be modelled. An eye 2 comprises a cornea 4, a pupil 6, a crystalline lens 8 and a retina 10.

The cornea 4 and the crystalline lens 8 act as lenses which concentrate the light rays, the pupil 6 acts as a diaphragm, and the retina 10 acts as a photoreceptor. Ideally, the cornea 4 is prolate and is at such a distance from the retina 10 that all images are formed in a focused manner on the retina 10 (zero spherical aberrations).

That is generally not the case. As can be seen in FIG. 2, there are three main types of corneal profile:
the prolate profile, for which the keratometric index is slightly greater at the centre than at the periphery, which induces an asphericity Q<0, with single-line hatching in FIG. 2,
the spherical profile, for which the keratometric index is constant over the eye (Q=0), and
the oblate profile, for which the keratometric index is slightly lower at the centre than at the periphery, which induces an asphericity Q>0, with double-lined hatching in FIG. 2.

In general, a prolate or slightly hyper-prolate profile is preferred because it permits better near vision. An oblate profile penalises distance vision, in particular night vision.

The crystalline lens 8 complements the cornea 4 and undergoes deformations in order to permit accommodation for near vision and for distance vision. The cornea 4 and the crystalline lens 8 can in fact be seen as a focusing system 12, the profile of which is generally prolate, spherical or oblate.

Myopia and hypermetropia are two ophthalmological conditions which result in distorted vision. In the case of myopia, the eye is too long and the retina 10 is disposed after the focal plane of the focusing system. The rays corresponding to distant images are accordingly not focused correctly and distance vision is not clear. In the case of hypermetropia, the opposite is true: the eye is too short. However, in this case, the accommodation of the crystalline lens may partly compensate for this defect. Another ophthalmological condition is presbyopia.

As people grow older, or as a result of some traumas, the crystalline lens 8 can undergo gradual opacification, which is also known by the name cataract. In addition, from the age of 40, the human eye gradually loses its ability to accommodate (contract) in order to deform the crystalline lens, which is necessary for clarity in near vision (loss of accommodation).

Cataract is a disorder which has been known since ancient times and which is nowadays treated successfully by means of a surgical operation during which the crystalline lens 8 is replaced by an intraocular lens or implant.

In order to take account of pre-existing vision problems in the patient, various types of implant have been developed, especially to correct myopia or hypermetropia. Nevertheless, these implants result in a considerable loss of quality in terms of near vision.

The situation is even worse when the focusing system has an oblate profile. In order to compensate for presbyopia, it is possible to add a magnifying lens, but this is cumbersome. It therefore appears that it is not possible at present to treat both myopia and presbyopia with an intraocular lens, or even to treat one of the two in isolation without penalising either the near vision or the distance vision. The only intraocular lenses that exist for that purpose are called “diffractive multifocals” and they use the principle of the Augustin Fresnel (1788-1827) lens described in 1822, which principle, apart from apodisation, has scarcely been improved.

This type of lens comprises a plurality of “steps”, each step acting like a prism which separates the light by means of two foci: one for distance vision and the other for near vision. Because the lens must be in one piece, the prisms are joined together by a continuity portion, and this dichotomy induces annoying light halos, a loss of contrast and/or a considerable defect in terms of intermediate vision.

Other methods consist in using an intraocular lens that treats near vision for one eye and an intraocular lens that treats distance vision for the other eye. These treatments produce a bascule called monovision. However, this does not give satisfactory results.

The Applicant's work has led him to study corneal profiles for their treatment by laser. More precisely, the Applicant has found that a corneal profile can be calculated for treating problems associated with near vision without affecting distance vision.

A simplified explanation is that this treatment will produce a corneal profile which is worked principally at the periphery, with a slightly prolate eye. The resulting asphericity is advantageously used to improve near vision, while distance vision is not affected because it is exerted mainly in the centre of the eye. This process is called “advanced isovision” and, unlike monovision, allows each eye to have excellent vision, both distance vision in a refractive manner and near vision in an aspherical manner.

In fact, referring to the Zernike polynomials:
distance vision will be corrected refractively by modifying the coefficient C4, or Z(2,0), called 1st defocus belonging to the 2nd order polynomial, and
intermediate and near vision will be corrected aspherically by virtue of the negative asphericity of the cornea, which induces negative spherical aberrations of coefficient C12 or Z(4,0), called 2nd defocus belonging to the 4th order polynomial.

It is therefore possible to use two types of optical correction, distance and near, which use different polynomial orders, of level two Z(2,0) of polar equation (2p²−1), and of level four Z(4,0) of polar equation (6p⁴−6p²+1). These corrections are therefore not in competition but, on the contrary, are complementary.

Such an optical system does not divide the light in two and allows monocular 20/20 J1 vision to be achieved, without compromising in terms of either distance vision or near vision or intermediate vision, and without any loss of contrast.

While pursuing this research, the Applicant extended his work to intraocular lenses and discovered how they can be profiled in order to treat both near vision and distance vision.

FIG. 3 shows an axial schematic view of an eye in which an intraocular lens 12 according to the invention has been implanted.

As will be seen hereinbelow, the profile of the intraocular lens 12 depends on the corneal profile of the eye 2 and on the general characteristics of the eye, such as its length etc. As will also become apparent, the profile of the intraocular lens 12 depends on a parameter called the “useful optical zone”.

In fact, when it is implanted, the intraocular lens 12 virtually comes into contact with the pupil 6, like the natural crystalline lens 8 which is usually situated in the posterior chamber, at a small distance of approximately 100 μm from the pupil 6. Owing to its positioning against the pupil 6, only a limited part, called the useful optical zone, will be passed through by light rays.

The useful optical zone of the intraocular lens 12 depends directly on the state of dilation of the pupil 6. The more the pupil 6 is dilated, the larger the useful optical zone.

In FIG. 3, the pupil 6 has been shown in its state of maximum dilation, or scotopic pupil. In this configuration, the diameter of the pupil is denoted Ps. In FIG. 4, the pupil 6 has been shown in its state of moderate dilation, or mesopic pupil. In this configuration, the diameter of the pupil is denoted Pm. In FIG. 5, the pupil 6 has been shown in its state of minimal dilation, or photopic pupil. In this configuration, the diameter of the pupil is denoted Pp.

Each of these states can be related to a sight condition. When it is night, light is minimal and the pupil 6 will therefore be dilated between Pm and Ps. Conversely, in broad daylight, light is maximum and the pupil 6 will therefore be dilated between Pm and Pp. For reasons which are quite evident, reading is generally associated with this latter case, that is to say when the pupil 6 is dilated between Pm and Pp. Consequently, the intraocular lens 12 has a profile optimised to function between Pm and Pp.

Before a cataract operation, the patient undergoes various tests, also called biometry. Biometry is carried out in order to determine a parameter of the intraocular lens called power. This parameter is used especially to choose an implant that is adapted to the structure of the patient's eye and allows his distance vision, for example, to be corrected.

In fact, the power of the implant is based on its anterior and posterior radii of curvature, its thickness and its refractive index n. The index n is peculiar to the material of which the implant is composed and is determined relative to a saline solution of refractive index 1.336, at 35° C., for a wavelength of 546.1 nm, which corresponds to the average wavelength of the spectrum perceived by the human eye.

The power is assessed over an optical zone of 3 mm in diameter. The radius of curvature at the centre of the intraocular lens 12 corresponding to this nominal power will be denoted Rc in the following. The power can be calculated, for example, by means of a formula of type SRK, which calculates it from a constant A dependent on the implant, the length L of the eye and the central keratometric index of the patient's cornea.

Many other formulae may be used to calculate the power as a function of the particular therapeutic indications of each patient, and therefore enable the equivalent radius of curvature Rc to be obtained. Once the nominal power has been determined, the radius of curvature Rc is fixed, since it is the radius of curvature at the centre of the intraocular lens which has the nominal power.

During this work on laser surgery, the Applicant has found that, in order to obtain optimal simultaneous treatment of myopia/hypermetropia and presbyopia, it is necessary to obtain a central index for the focusing system which corrects myopia/hypermetropia, and to modulate the off-centre profile relative to the optical axis in order to obtain an asphericity value Q which depends on the age of the patient. This is described in French patent application FR 11/02842.

In the present case, since the intraocular lens will replace the crystalline lens, there is no longer any accommodation at all. The target asphericity is therefore fixed and can have a necessary and sufficient value such as −1.0. And, as has been seen above, this target asphericity value must be obtained for the mesopic pupil.

The Applicant has therefore created intraocular lenses whose radius of curvature profile is such that, in a central zone, the power of the intraocular lens is the nominal power taken from the biometry and which corresponds to the radius of curvature Rc and, in a peripheral zone, at a distance corresponding to the mesopic pupil, the radius of curvature is such that the asphericity is −1.0. In general, the distance at which the asphericity obtained must be equal to −1.0 will be called the emmetropic distance and denoted De.

As will be seen below, the distance De is an important parameter for the intraocular lens since it indirectly defines its radius of curvature profile. In general, the distance De depends on the mesopic pupil Pm. By way of variation, the distance De may be calculated from a function having as argument the mesopic pupil Pm, as well as the photopic pupil Pp and/or the scotopic pupil Ps. In the examples described with FIGS. 6 to 8, the distance De is equal to Pm/2. In the following, the distances, whether they relate to Ps, Pm, Pp or De, or another distance, are given in mm, according to the axis x, which is perpendicular to the optical axis y.

In FIGS. 6 to 8, the profiles shown are based on the following parameters:
Pp=1 mm,
De=Pm/2=3 mm,
Rc=23 dioptres,
Rp=17 dioptres, and
α=0.5.

FIG. 6 shows a first preferred radius of curvature profile for an intraocular lens according to the invention.

In this embodiment, the radius of curvature of the intraocular lens 12 varies according to four zones denoted Z1, Z2, Z3 and Z4.

In the example described here, zone Z1 comprises the part of the intraocular lens according to the axis x which is contained in the range [−Pp/2; Pp/2]. Zone Z1 in fact corresponds to the zone of the intraocular lens which is used for distance vision. In zone Z1, the radius of curvature of the intraocular lens is equal to the radius of curvature Rc. Distance vision is thus ensured.

In the example described here, zone Z2 comprises the part of the intraocular lens which is contained according to the axis x in the ranges [−Dc; −Pp/2] and [Pp/2; Dc], that is to say [−Pm/2; −Pp/2] and [Pp/2; Pm/2]. Zone Z2 in fact corresponds to the zone of the intraocular lens 12 which is contained between the photopic pupil Pp and the mesopic pupil Pm, that is to say the zone which is used for reading or near vision in general.

As has been seen above, the desired aim is that the asphericity Q is equal to −1.0 at distance De. To that end, the intraocular lens must have a radius of curvature Rp which can be calculated from formula [10] of Annex A.

In zone Z2, the radius of curvature of the intraocular lens is therefore equal to Rc for x equal to −Pp/2 and to Pp/2, and to Rp for x equal to −Pm/2 and Pm/2. Between those values, the Applicant has found that it is advantageous for the radius of curvature of the intraocular lens in zone Z2 to evolve according to formula [20] of Annex A. This profile in fact allows the desired asphericity to be obtained gradually.

In the example described here, zone Z3 comprises the part of the intraocular lens which is contained according to axis x in the ranges [−(2De−Pp/2); −De] and [De; (2De−Pp/2)], that is to say [−(Pm−Pp/2); −Pm/2] and [Pm/2; (Pm−Pp/2)]. Zone Z3 in fact corresponds to the zone of the intraocular lens which is contained between the photopic pupil Pm and the scotopic pupil Ps, that is to say the zone of the pupil which is used for night vision.

The Applicant has found that it is advantageous for the radius of curvature of the intraocular lens in zone Z3 to evolve according to formula [30] of Annex A. In fact, this matches the profile of the intraocular lens with zone Z2.

Finally, zone Z4 comprises, in the example described here, the part of the intraocular lens which is contained according to axis x in the ranges [−6.5; −(2De−Pp/2)] and [(2De−Pp/2); 6.5], that is to say [−6.5; −(Pm−Pp/2)] and [(Pm−Pp/2); 6.5]. Zone Z4 in fact corresponds to the part of the intraocular lens which is not exposed to light.

The Applicant has found that it is advantageous for the radius of curvature of the intraocular lens to be equal to 2Rp−Rc in zone Z4, that is to say the radius of curvature of the intraocular lens at the end of zone Z3.

FIG. 7 shows another embodiment of the intraocular lens according to the invention. In this embodiment, the Applicant has considered that the progression in zone Z3 should be reduced so that the asphericity does not diminish too greatly. Zones Z1 to Z4 and the values of Rc and Rp have not been shown because they are identical to those of FIG. 6.

To that end, the radius of curvature of the intraocular lens in zone Z3 evolves according to formula [30] of Annex A, where the coefficient α is a real number in the range ]0; 1[ and is chosen in that range, for example as a function of a ratio C of formula [40] of Annex A. In order to preserve the continuity, the radius of curvature of the intraocular lens in zone Z4 is identical to the radius of curvature of the intraocular lens at the end of zone Z3, that is to say it is greater than in the case of FIG. 6. In practice, this value is equal to (1+α)Rp−Rc.

FIG. 8 shows yet another embodiment of the intraocular lens according to the invention. In this embodiment, the Applicant has simplified the radius of curvature profile of the intraocular lens, so that:
the radius of curvature in zones Z1 and Z4 is identical to that of the lens of FIG. 6,
the radius of curvature evolves linearly in zones Z2 and Z3, and
the radius of curvature is equal to Rp for x equal to De and −De, that is to say −Pm/2 and Pm/2.

In a variant of this embodiment, zone Z3 and zone Z4 can be merged and have a radius of curvature equal to Rp, with the same aim as that pursued with the embodiment of FIG. 7. For the sake of simplicity, zones Z1 to Z4 and the values Rc and Rp have also not been shown in this figure.

In the above embodiments, zone Z1 can be extended or reduced in width, and zone Z3 can likewise be extended or omitted, or merged with zone Z2 or zone Z4. Zone Z4 can further be delimited not by the value x equal to 2De−Pp/2 but by the value x equal to Ps. In this case, the formulae of Annex A will be adapted. Finally, functions other than the function cos( ) may be used. It is particularly apparent from these embodiments that the radius of curvature can be described by a continuous mathematical function, the values of which are between Rc and Rp at least.

FIG. 9 shows a schematic flow diagram of a method for manufacturing an intraocular lens according to one of the above embodiments.

This method starts by an operation 900 in which parameters relating to the patient are received. These parameters are the desired radius of curvature Rc at the centre of the intraocular lens or the corresponding nominal power, as well as at least the distances Pp and Pm of the patient. By way of variation, the distance Ps can also be received.

Then, in an operation 910, the emmetropic distance De is calculated, either by defining it as equal to Pm/2, or by a function of the distances Pm, as well as Pp and/or Ps. The operation 910 also includes the calculation of the radius of curvature Rp which allows an asphericity value of −1.0 to be obtained at distance −De/2 and De/2.

Once operation 910 is complete, the radius of curvature profile of the intraocular lens is calculated in an operation 920, according to one of the profiles described with FIGS. 6 to 8, and by definition of the various zones Z1 to Z4.

Finally, in an operation 930, the intraocular lens is manufactured according to the profile calculated in operation 920.

It is apparent that the method of FIG. 9 includes a method for calculating the radius of curvature profile of an intraocular lens and a manufacturing step based on that profile.

FIG. 10 shows a simplified diagram of a device 20 for calculating a radius of curvature profile of an intraocular lens according to the invention.

The device 20 comprises a memory 24, a processing unit 26, an interface 28 and a scheduler 30.

In the example described here, the memory 24 is a conventional storage medium, which can be a hard disk with platters or flash memory (SSD), flash memory or ROM, a physical storage medium such as a compact disk (CD), a DVD disk, a Blu-Ray disk, or any other type of physical storage medium. The storage unit 24 can also be remote, on a storage area network (SAN), or on the internet, or generally in the “cloud”.

In the example described here, the processing unit 26 is a software element executed by a computer which contains it. However, it may also be executed in a distributed manner on a plurality of computers or be realised in the form of a printed circuit (ASIC, FPGA or the like) or of a dedicated single-core or multi-core microprocessor (NoC or SoC).

The interface 28 allows a practitioner to enter the biometric parameters relating to a patient for whom the radius of curvature profile calculation is desired, and to adjust some of those parameters if required. The interface 28 can be electronic, that is to say it can be a connection between the device 20 and another piece of equipment allowing the practitioner to interact with the device 20. The interface 28 can also include such a piece of equipment and comprise, for example, a display and/or loudspeakers in order to permit communication with the practitioner.

The scheduler 30 selectively controls the processing unit 26 and the interface 28, and accesses the memory 24 in order to carry out the processing operations of the method of FIG. 9.

It is clear from the above that the Applicant has found an intraocular lens whose radius of curvature profile allows myopia/hypermetropia, astigmatism and presbyopia to be treated simultaneously. This is obtained by defining a continuous and monotonous (strictly or in the broad sense) radius of curvature profile which associates two radius of curvature values (Rc and Rp), one of which (that corresponding to Rc) corresponds to a nominal optical power determined in the conventional manner.

Accordingly, the radius of curvature profile comprises a central zone (Z1), in which the optical power is nominal, and a peripheral zone (Z2, Z3, Z4), in which the optical power varies, so that a target asphericity value (−1.0) is obtained at a chosen distance (De) from the optical axis. In the peripheral zone, zone Z2 can be seen as an emmetropic zone, zone Z3 as an intermediate zone and zone Z4 as an end zone, zones Z3 and Z4 defining between them an external zone.

Unlike diffractive lenses, the profile so defined does not require a continuity solution, or a step, and consequently does not therefore induce halos or losses of contrast. The spherical aberrations that are produced are in fact like an optical property added to the refractive characteristic, given by the central power of the implant, and they are created by peripheral reduction of the radius of curvature of the implant.

That is obtained especially by the use of optical effects that are not utilised in known intraocular lenses. In fact, until the finding made by the Applicant, it was considered that only the 2nd order Zernicke polynomials could be used.

It will be noted that the lens of the invention has been described with the aim of obtaining an asphericity equal to −1.0 at the second distance. In the more general case, if a different target asphericity value is desired, it is sufficient to change the value of the radius of curvature Rp at the second distance, according to formula [50] of Annex A.

In various alternatives, the device may have the following features:
the peripheral zone (Z2, Z3, Z4) comprises an emmetropic zone (Z2) extending between the first distance (Pp/2) and the second distance (De), wherein the radius of curvature varies continuously and strictly monotonously in the emmetropic zone (Z2),
the radius of curvature varies as a function of the distance to the optical axis according to an at least partly trigonometric function ([20]) in the emmetropic zone (Z2),
the radius of curvature varies linearly as a function of the distance to the optical axis in the emmetropic zone (Z2),
the peripheral zone (Z2, Z3, Z4) comprises an external zone (Z3, Z4) extending beyond the second distance (De), wherein the radius of curvature varies continuously and monotonously,
the radius of curvature varies as a function of the distance to the optical axis according to an at least partly trigonometric function ([20], [30]) in the external zone (Z3, Z4),
the radius of curvature varies linearly as a function of the distance to the optical axis in the external zone (Z3, Z4),
the radius of curvature is substantially constant in the external zone (Z3, Z4),
the external zone (Z3, Z4) comprises an intermediate zone (Z3) extending between the second distance (De/2) and a third distance (2De−Pp/2), and an end zone (Z4) extending between the third distance (De−Pp/2) and the end of the lens, the third distance (2De−Pp/2) being calculated from a mesopic pupil diameter (Pm) and a photopic pupil diameter (Pp) of a patient,
the radius of curvature varies as a function of the distance to the optical axis according to an at least partly trigonometric function ([20], [30]) in the intermediate zone (Z3),
the radius of curvature varies linearly as a function of the distance to the optical axis in the intermediate zone (Z3), and
the radius of curvature is substantially constant in the end zone (Z4).

It will be recalled that intraocular lenses are composed of a central portion called the “optic” of the implant, which serves to correct the vision over a diameter of from 6 to 6.5 mm, connected to a plurality of “haptics”, which serve to centre and stabilise the intraocular lens in the lenticular sac. The intraocular lenses can be single-piece or have attached struts, also called three-piece implants. The invention described above concentrates on the “optical” part of the lens and is therefore not limited to a specific type of haptic. In general, the invention relates to an intraocular lens which is spherical, or spherocylindrical for correcting associated astigmatism. The lens can be made of various types of hydrophilic, hydrophobic, liquid, etc. materials. By way of variation, the variation of the asphericity Q may be obtained not by varying the radius of curvature but by varying the index n of the material between its centre and its periphery. Moreover, other target Q values different from −1.00, such as −1.05 or −1.10 or the like, may likewise be obtained.

The invention relates also to a method for manufacturing an intraocular lens, wherein a radius of curvature profile is determined by the method for calculating a radius of curvature profile described above, and wherein an intraocular lens is manufactured in accordance with that radius of curvature profile.

### ANNEX A

$$\begin{matrix}Rp = \sqrt{3} * Rc \text{/} 2 & [ 10 ] \\ R ( x ) = Rp + \vert Rc - Rp \vert cos ( ( \frac{x}{2} * \frac{\vert s \vert Pp \text{/} 2}{( Pm - Pp ) / 2} ) ) & [ 20 ] \\ R ( x ) = Rp + α \vert Rc - Rp \vert cos ( ( \frac{x}{2} * \frac{\vert s \vert Pp \text{/} 2}{( Pm - Pp ) / 2} ) ) & [ 30 ] \\ C = \frac{\vert Rc - Rp \vert}{Rc + Rp} & [ 40 ] \\ Rp = \sqrt{4 + Q} * Rc \text{/} 2 & [ 50 ]\end{matrix}$$

1. Intraocular lens, characterised in that it has an optical axis (y), a central zone (Z1), and a peripheral zone (Z2, Z3, Z4) which are substantially symmetrical with respect to said optical axis (y) and which extend substantially perpendicular thereto, said central zone (Z1) extending to a first distance (Pp/2) and the peripheral zone (Z2, Z3, Z4) extending from the first distance (Pp/2) to the end of the intraocular lens, wherein the central zone (Z1) has a nominal optical power, and the peripheral zone (Z2, Z3, Z4) has a radius of curvature which varies continuously and monotonously as a function of the distance (x) to the optical axis (y), so that a target asphericity value is obtained at a second distance (De) relative to the optical axis (y), the first distance (Pp/2) and the second distance (De) being calculated from a photopic pupil diameter (Pp) and a mesopic pupil diameter (Pm), respectively, of a patient.

2. Intraocular lens according to claim 1, wherein the peripheral zone (Z2, Z3, Z4) comprises an emmetropic zone (Z2) which extends between the first distance (Pp/2) and the second distance (De), wherein the radius of curvature varies continuously and strictly monotonously in the emmetropic zone (Z2).

3. Intraocular lens according to claim 2, wherein the radius of curvature varies as a function of the distance to the optical axis according to an at least partly trigonometric function ([20]) in the emmetropic zone (Z2).

4. Intraocular lens according to claim 2, wherein the radius of curvature varies linearly as a function of the distance to the optical axis in the emmetropic zone (Z2).

5. Intraocular lens according to claim 1, wherein the peripheral zone (Z2, Z3, Z4) comprises an external zone (Z3, Z4) which extends beyond the second distance (De), wherein the radius of curvature varies continuously and monotonously.

6. Intraocular lens according to claim 5, wherein the radius of curvature varies as a function of the distance to the optical axis according to an at least partly trigonometric function ([20], [30]) in the external zone (Z3, Z4).

7. Intraocular lens according to claim 5, wherein the radius of curvature varies linearly as a function of the distance to the optical axis in the external zone (Z3, Z4).

8. Intraocular lens according to claim 5, wherein the radius of curvature is substantially constant in the external zone (Z3, Z4).

9. Intraocular lens according to claim 5, wherein the external zone (Z3, Z4|) comprises an intermediate zone (Z3) which extends between the second distance (De/2) and a third distance (2De−Pp/2) and an end zone (Z4) which extends between the third distance (De−Pp/2) and the end of the lens, the third distance (2De−Pp/2) being calculated from a mesopic pupil diameter (Pm) and a photopic pupil diameter (Pp) of a patient.

10. Intraocular lens according to claim 9, wherein the radius of curvature varies as a function of the distance to the optical axis according to an at least partly trigonometric function ([20], [30]) in the intermediate zone (Z3).

11. Intraocular lens according to claim 9, wherein the radius of curvature varies linearly as a function of the distance to the optical axis in the intermediate zone (Z3).

12. Intraocular lens according to claim 9, wherein the radius of curvature is substantially constant in the end zone (Z4).

13. Method for calculating a radius of curvature profile for an intraocular lens, characterised in that it comprises the following steps:
a) receiving biometric parameters of a patient, comprising at least a first radius of curvature (Rc), a photopic pupil diameter (Pp) and a mesopic pupil diameter (Pm),
b) determining an emmetropic distance (De) from at least the mesopic pupil diameter (Pm), and a second radius of curvature (Rp) from the first radius of curvature (Rc) and a target asphericity value,
c) calculating for the intraocular lens a desired radius of curvature profile in a direction substantially perpendicular to an optical axis (x), wherein the radius of curvature is equal to the first radius of curvature (Rc) in a central zone (Z1) extending between the optical axis (y) and a first distance (Pp/2) calculated from at least the photopic pupil diameter (Pp), and wherein, in a peripheral zone (Z2, Z3, Z4) extending from the first distance (Pp/2) to the end of the intraocular lens, the radius of curvature varies continuously and monotonously as a function of the distance (x) to the optical axis (y), so that the radius of curvature is equal to the second radius of curvature (Rp) at the emmetropic distance (De) relative to the optical axis (y).

14. Method for manufacturing an intraocular lens, wherein a radius of curvature profile is determined by the method of claim 13, and wherein an intraocular lens is manufactured in accordance with that radius of curvature profile.

15. Intraocular lens according to claim 2, wherein the peripheral zone (Z2, Z3, Z4) comprises an external zone (Z3, Z4) which extends beyond the second distance (De), wherein the radius of curvature varies continuously and monotonously.

16. Intraocular lens according to claim 3, wherein the peripheral zone (Z2, Z3, Z4) comprises an external zone (Z3, Z4) which extends beyond the second distance (De), wherein the radius of curvature varies continuously and monotonously.

17. Intraocular lens according to claim 4, wherein the peripheral zone (Z2, Z3, Z4) comprises an external zone (Z3, Z4) which extends beyond the second distance (De), wherein the radius of curvature varies continuously and monotonously.

18. Intraocular lens according to claim 10, wherein the radius of curvature is substantially constant in the end zone (Z4).

19. Intraocular lens according to claim 11, wherein the radius of curvature is substantially constant in the end zone (Z4).
